(19) United States
(12) Patent Application Publication
(10) Pub. No.: US 2014/0243701 A1
(43) Pub. Date: Aug. 28, 2014

(54) Temperature Measurement System and Method

(71) Applicant: RESONANT CIRCUITS LTD, London (GB)

(72) Inventors: Paul Southern, London (GB); Simon Hattersley, Bickley (GB)

(73) Assignee: RESONANT CIRCUITS LTD, London (GB)

(21) Appl. No.: 14/186,465

(22) Filed: Feb. 21, 2014

Related U.S. Application Data
(60) Provisional application No. 61/768,020, filed on Feb. 22, 2013.

(51) Int. Cl. A61B 5/01

(52) U.S. Cl. CPC A61B 5/01
USPC 600/549

(57) ABSTRACT

A method and apparatus for measuring the change in temperature of a material being heated. In one embodiment, the method includes introducing particles having magnetic susceptibility into the material; heating the material and particles; measuring, using a circuit having a resonant frequency, the change in the resonant frequency of the circuit as the temperature of the particles changes; and correlating the change in resonant frequency to a change in temperature. In one embodiment, the apparatus includes a collection of magnetic particles in contact with the material undergoing heating; a resonance circuit including a tank circuit including a coil and capacitance and having a resonant frequency; an alternating current variable frequency source capable of tracking changes in the resonant frequency of the tank circuit to maintain the frequency of the alternating current at the resonant frequency of the tank circuit; and a processor in communication with the alternating current source.

### RELATED APPLICATIONS

This application claims priority to U.S. Provisional Application No. 61/768,020 filed on Feb. 22, 2013, the contents of which are herein incorporated by reference in their entirety.

### FIELD OF INVENTION

This invention relates to the field of temperature measurement and more specifically to the measurement of temperature without contact with the object being measured.

### BACKGROUND

Thermotherapeutic treatments of disease such as cancer function by causing the temperature of a target tissue to increase to a level which causes the target tissue to change physiologically or die. The effectiveness of the technique relies upon accurate measurement of temperature in the target tissue. Too small a temperature increase, and the treatment may not be effective. Too large a temperature increase, and the temperature of non-intended tissues may rise.

The accurate measurement of temperature is difficult, especially in tissues located deep within the body. Standard techniques such as infrared or other optical probes are limited substantially to accessible tissue surfaces of the body. What is needed is a method of accurately measuring temperature in a tissue located deep within the body without using invasive means.

The present invention addresses this need.

### SUMMARY OF THE INVENTION

In one aspect, the invention relates to a method for measuring the change in temperature of a material being heated. In one embodiment, the method includes the steps of introducing particles having one or both of a magnetic susceptibility and an electrical conductivity into the material; heating the material and particles; measuring, using a circuit having a resonant frequency, the change in the resonant frequency of the circuit as the temperature of the particles changes; and correlating the change in resonant frequency to a change in material temperature. In another embodiment, the particles are nanoparticles. In still another embodiment, the nanoparticles are magnetic.

In yet another embodiment, the material is a tumor and the particles comprise a nanoparticle and an antibody or receptor. In still yet another embodiment, the circuit is a tank circuit in electrical communication with a variable alternating current source. In one embodiment, the variable alternating current source adjusts the frequency of the alternating current to track the resonant frequency as the resonant frequency changes. In another embodiment, the step of correlating comprises relating the change in resonant frequency to a temperature change using a calibration table or function by a computer. In yet another embodiment, the heating of the material and the measuring of the resonant frequency change is performed with the same circuit.

In another aspect, the invention relates to a method of measuring temperature in a tissue undergoing hyperthermia treatment. In one embodiment, the method includes the steps of: introducing magnetic and/or conductive particles to the tumor; measuring, using a circuit which has a coil in a tank circuit and a resonant frequency, the change in resonant frequency of the circuit during hyperthermia treatment; tracking a change in electromagnetic resonant frequency during hyperthermia treatment; and correlating the change in electromagnetic resonant frequency to a change in tissue temperature using a computing device monitoring the circuit. In another embodiment, the particles are nanoparticles. In still another embodiment, the nanoparticles are magnetic. In yet another embodiment, the particles include a nanoparticle and an antibody or receptor ligand. In still yet another embodiment, the tank circuit is in electrical communication with an alternating current variable frequency source.

In one embodiment, the alternating current variable frequency source adjusts the frequency of the alternating current to track the resonant frequency as the resonant frequency of the circuit changes. In another embodiment, the step of correlating comprises relating, using a computing device, the change in resonant frequency to a temperature change using a calibration table or function. In yet another embodiment, the hyperthermia treatment and the measuring of the resonant frequency change is performed with the same circuit.

In another aspect, the invention relates to a system for measuring the temperature change in a material. In one embodiment, the system includes a magnetic and/or conductive particle in contact with the material undergoing heating; a resonance circuit comprising: a tank circuit comprising inductance and capacitance and having a resonant frequency; an alternating current variable frequency source capable of tracking changes in the resonant frequency of the tank circuit to maintain the frequency of the alternating current at the then current resonant frequency of the tank circuit; and a processor in electrical communication with the alternating current variable frequency source for measuring the change in resonant frequency of the tank circuit in response over time. In one embodiment, the tank circuit generates an alternating magnetic field in response to current from the alternating current variable frequency source. In another embodiment, when the magnetic or conductive particles are within the alternating magnetic field, the resonant frequency of the tank circuit changes. In still yet another embodiment, the temperature of the magnetic or conductive particles causes the resonant frequency of the tank circuit to change. In one embodiment, the particles are nanoparticles. In another embodiment, the nanoparticles are bound to an antibody or receptor ligand.

This Summary is provided merely to introduce certain concepts and not to identify any key or essential features of the claimed subject matter.

### BRIEF DESCRIPTION OF DRAWINGS

The structure and function of the invention can be best understood from the description herein in conjunction with the figures. The figures are not necessarily to scale, emphasis instead generally being placed upon illustrative principles. The figures are to be considered illustrative in all aspects and are not intended to limit the invention, the scope of which is defined only by the claims.

FIG. 1 is a highly schematic diagram of an embodiment of the system constructed in accordance with the invention;

FIG. 1(a) is a highly schematic diagram of the embodiment of the system of FIG. 1 with a probe used to calibrate the changes in magnetization with temperature;

FIG. 2 is a plot of the magnetization of a magnetic material versus applied magnetic field at different temperatures for high magnetic fields;

FIG. 3 is a plot of the magnetic saturation of a magnetic material versus temperature;

FIG. 4 is a graph of the low field magnetization of a magnetic material for two different temperatures; and

FIG. 5 is a graph of resonant frequency and temperature measured with a fluoroptic probe plotted against time.

### DETAILED DESCRIPTION

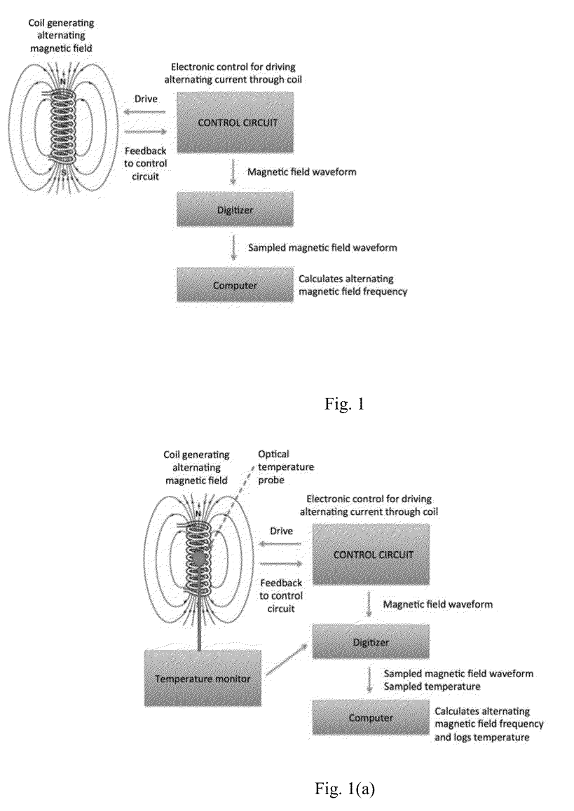

In brief overview and referring to FIG. 1, a system for measuring temperature includes a coil having self-inductance and a capacitor (not shown) constructed in parallel as an LC “tank” circuit. An electronic control unit supplies an alternating current through the coil. The inductance of the coil (L) and the capacitance of the capacitor (C) react oppositely to the alternating current and as a result, the combination of the value of capacitance of the tank circuit and inductance of the tank circuit produces a natural resonant frequency of tank circuit described by the equation:

f=a{1/(LC)^{1/2}}

where f is the resonant frequency and (a) is the constant of proportionality.

As the inductance (L) or the capacitance (C) changes, the resonant frequency will therefore change. By tracking the change in resonant frequency, one can track the change in the components of the circuit. When a magnetic or conductive material enters the field generated by the coil, the magnetic field changes and this in turn affects the self-inductance of the coil and hence the resonant frequency of the system, i.e. the combination of tank circuit and magnetic material. This change in the magnetic field generated by the coil is a result of the magnetization of the magnetic material or the flow of eddy currents in the conductive material.

The effect of the magnetic material on the inductance of the coil is a function of the material's magnetic susceptibility. The magnetic material's magnetic susceptibility varies with temperature. Thus, as the temperature of the material changes, the susceptibility changes, resulting in a change in the coil inductance and thus a change in the resonant frequency of the system.

In the case of a collection of magnetic nanoparticles of a single size, theory states that the magnetic susceptibility at low fields is inversely proportional to the absolute temperature. For instance, a 50K increase from room temperature in theory could result in a change in susceptibility of up to 17%. In real preparations, magnetic nanoparticles have a distribution of sizes which makes it necessary to calibrate the magnetic susceptibility against the temperature.

FIG. 1(a) is a highly schematic diagram of the system of FIG. 1 but with an optical temperature probe to measure, by another means, the temperature of the tissue being measured using magnetic susceptibility. With this probe, the change in frequency can be correlated with the change in temperature and the system calibrated. Once calibrated, the optical temperature probe is removed from the system.

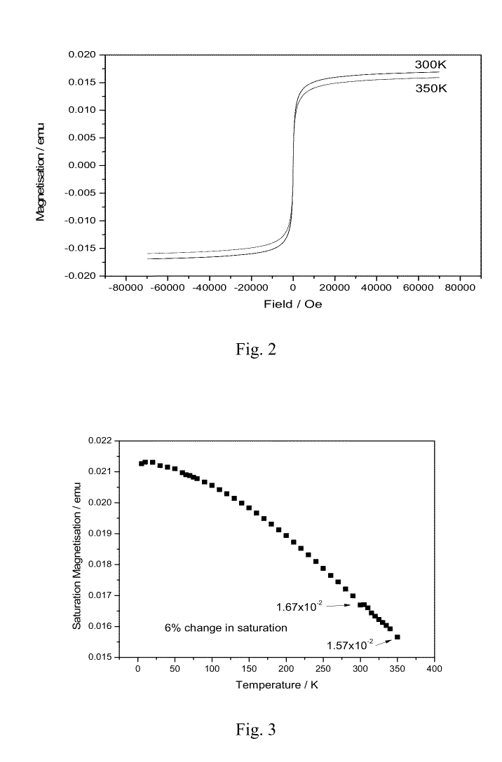

FIG. 2 depicts the change of magnetization of a magnetic material plotted against applied magnetic field at two different temperatures. It can be seen that the magnetization of a magnetic material increases with increasing applied field but decreases with increasing temperature. FIG. 3 depicts how the magnetic saturation of a magnetic material decreases with increasing temperature.

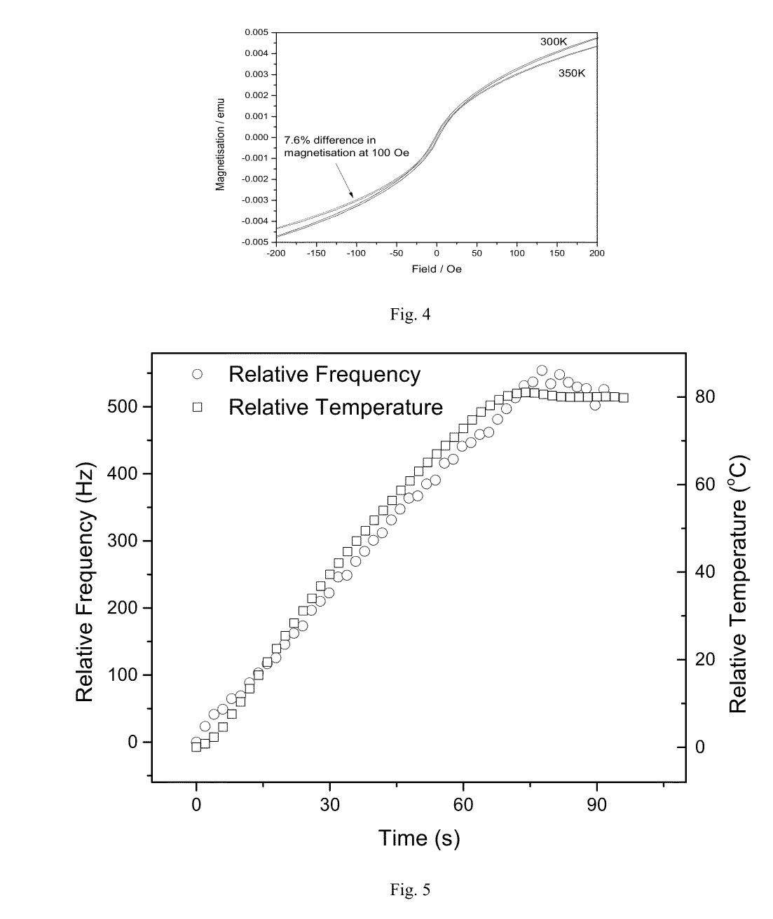

Referring to FIG. 4, it is important to note that the change in magnetization with temperature is a function of the field strength. FIG. 2 depicts this change with strong fields, while FIG. 4 depicts the change with the field strengths typically used in measurements made, for example, on a human body.

By tracking the change in resonant frequency, one can determine the change in the temperature of the magnetic or conductive material being measured. FIG. 5 shows the change in resonant frequency of the system and the change in temperature of the magnetic material, as measured by an optical probe, plotted against time, as a magnetic material is heated. In this example, the measurement and the heating is performed with the same device. That is, the material is heated using an induction heater that maintains the frequency of the alternating current supplied to the coil at the resonant frequency of the system. It is easy to note that there is an almost proportional change in resonant frequency to the change in temperature of the magnetic material. Although in this case the induction heating and the resonance measurement were accomplished with the same device, the measurement in the change in temperature of the material may be made separately from the device causing the heating of the material.

The electronic control unit which provides the current to the tank circuit includes alternating current variable frequency source and a feedback loop and varies the frequency of the alternating current to compensate for the change in resonant frequency of the tank circuit. The output of the control unit is connected to the input of a processor and transmits the magnetic field strength and the alternating current frequency at which the field strength is measured.

The processor compares the frequency changes against a table of values listing the change in temperature against a change in frequency for a given type of magnetic or conductive particle to determine the effective temperature change. The resonance tracking circuit of the controller is of standard configuration and is well known to one skilled in the art. The coil is shown as a tube but the coil may in fact be any inductor regardless of shape, such as a flat or plate coil.

In use, the magnetic or conductive material, in one embodiment, is a magnetic nanoparticle of iron oxide coated with a dextran such as Ferucarbotran (Meito Sangyo Ltd, Nagoya, Japan). Generally these particles are used for the thermotherapeutic treatment of cancer. The magnetic nanoparticle is frequently coupled to antibodies or receptor ligands which will bind to an antigen or receptor in the cell membrane of the cancer cells. In one embodiment, the magnetic particles are conjugated using sodium periodate to sm3E, a single chain Fv antibody fragment which binds to human carcinoembryonic antigen (CEA4,5). In other embodiments, for example for use with glioblastoma, the magnetic particles are bound to Designed Ankyrin Repeat Proteins. When introduced into the body, the magnetic nanoparticle-antibody complex binds the magnetic nanoparticles to the cancer. The cancerous tumor then is heated by induction heating as described above.

Using the technique described, the shift in the resonant frequency of the system provides a measure of the change in temperature of the magnetic nanoparticles and hence the tumor to which they are bound. In this way, it is possible to assure that the temperature of the tumor has risen sufficiently to be damaged or killed.

The aspects, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

What is claimed is:

1. A method for measuring the change in temperature of a material being heated, the method comprising the steps of:
introducing particles having one or both of a magnetic susceptibility and an electrical conductivity into the material;
heating the material and particles;
measuring, using a circuit having a resonant frequency, the change in the resonant frequency of the circuit as the temperature of the particles changes; and
correlating the change in resonant frequency to a change in material temperature.

2. The method of claim 1 wherein the particles are nanoparticles.

3. The method of claim 2 wherein the nanoparticles are magnetic.

4. The method of claim 1 wherein the material is a tumor and the particles comprise a nanoparticle and an antibody or receptor.

5. The method of claim 1 wherein the circuit is a tank circuit in electrical communication with a variable alternating current source.

6. The method of claim 5 wherein the variable alternating current source adjusts the frequency of the alternating current to track the resonant frequency of the circuit as it changes.

7. The method of claim 1 wherein the step of correlating comprises relating the change in resonant frequency to a temperature change using a calibration table or function.

8. The method of claim 1 wherein the heating of the material and the measuring of the resonant frequency change is performed with the same circuit.

9. A method of measuring temperature in a tissue undergoing hyperthermia treatment comprising the steps of:
introducing magnetic and/or conductive particles to the tumor;
measuring, using a circuit which has at least one coil in a resonant circuit, the change in resonant frequency of the circuit during hyperthermia treatment;
tracking a change in electromagnetic resonant frequency during hyperthermia treatment; and
correlating the change in electromagnetic resonant frequency to a change in tissue temperature using a computing device monitoring the circuit.

10. The method of claim 9 wherein the particles are nanoparticles.

11. The method of claim 10 wherein the nanoparticles are magnetic.

12. The method of claim 9 wherein the particles comprise a nanoparticle and an antibody or receptor.

13. The method of claim 9 wherein the tank circuit is in electrical communication with an alternating current variable frequency source.

14. The method of claim 13 wherein the alternating current variable frequency source adjusts the frequency of the alternating current to track the resonant frequency of the tank circuit as the resonant frequency of the tank circuit changes.

15. The method of claim 9 wherein the step of correlating comprises relating the change in resonant frequency to a temperature change using a calibration table or function.

16. The method of claim 1 wherein the hyperthermia treatment and the measuring of the resonant frequency change is performed with the same circuit.

17. A system for measuring the temperature change in a material comprising:
a magnetic and/or conductive particle in contact with the material under going heating;
a resonance circuit comprising:
a tank circuit comprising at least one coil and capacitance and having a resonant frequency;
an alternating current variable frequency source capable of tracking changes in the resonant frequency of the tank circuit to maintain the frequency of the alternating current at the current resonant frequency of the tank circuit; and
a processor in electrical communication with the alternating current variable frequency source for measuring the change in resonant frequency of the tank circuit over time,
wherein the tank circuit generates an alternating magnetic field in response to current from the alternating current variable frequency source;
wherein when the magnetic or conductive particles are within the alternating magnetic field, the resonant frequency of the tank circuit changes;
wherein the temperature of the magnetic or conductive particles causes the resonant frequency of the tank circuit to change.

18. The system of claim 17 wherein the particles are nanoparticles.

19. The system of claim 18 wherein the nanoparticles are bound to an antibody or receptor ligand.
